(19) United States
(12) Patent Application Publication
(10) Pub. No.: US 2006/0210483 A1
(43) Pub. Date: Sep. 21, 2006

(54) Non-systemic antibiotic formulations and related method of use and treatment of upper respiratory infections

(76) Inventor: Robert E. Kodsi, Brooklyn, NY (US)

Correspondence Address: ONOFRIO LAW, 107 SHAD ROW, PIERMONT, NY, 10968, US

(21) Appl. No.: 11/365,420

(22) Filed: Mar. 1, 2006

Related U.S. Application Data
(60) Provisional application No. 60/657,782, filed on Mar. 2, 2005.

(51) Int. Cl. A61K 31/496; A61L 9/04

(52) U.S. Cl. 424/45; 514/252.13

(57) ABSTRACT

A method of treatment and prevention of upper respiratory illnesses by airborne administration to a subject in need of such treatment a composition containing a therapeutically effective amount of a non-systemic antibiotic.

This application claims the benefit of U.S. provisional application No. 60/657,782 filed Mar. 2, 2005 which is incorporated herein in its entirety by reference.

### FIELD OF THE INVENTION

This invention relates to use of non-systemic antibiotic formulations in the prevention and treatment of upper respiratory infections caused by bacteria, mycobacterium, RNA dependent viruses, reverse transcriptase dependent viruses and any other infections etiology that utilizes RNA. In particular, the formulations are made to be airborne in a form which is inhaled by the subject to deliver the antibiotic to lung tissue.

### BACKGROUND OF THE INVENTION

In general, rifaximin is well known as a non-systemic antibiotic (<0.4%) characterized by activity against a broad spectrum of enteric bacterial pathogens and the delivery of high concentrations of antibiotic to the gastrointestinal tract.

The antibiotic rifaximin was discovered in 1980 and originally patented in Italy as IT Patent 1154655 granted on Jan. 21, 1987. The related U.S. Pat. No. 4,341,785 to Marchi et al. discloses imidazo-rifamicyn derivatives having antibacterial utility, and the related process for preparing it. The '785 patent also discloses a pharmaceutical antibacterial composition and a method of using it to treat antibacterial diseases of the gastrointestinal tract. A further patent, U.S. Pat. No. 4,557,866 to Cannata et al. discloses a process for the synthesis of pyrido-imidazo rifamycins. The process is described as an improvement over the '785 patent to Marchi in that the later process provides unsatisfactory yields from an industrial point of view.

Rifaximin is essentially a non-absorbable semi-synthetic antibiotic, related to rifamycin. The antimicrobial spectrum (in vitro) includes most gram-positive and gram-negative bacteria; and both aerobes and anaerobes.

It presents low risk for drug interactions (no effect on drugs metabolized by cytochrome p450 enzyme system) and about the same adverse properties as compared to a placebo. When ingested in tablet or pill form rifaximin is concentrated in the gastrointestinal tract and primarily excreted unchanged in the feces. It binds to the beta subunit of bacterial DNA-dependent RNA polymerase, which inhibits bacterial RNA synthesis. In contrast with other antibiotics, resistance to rifaximin is not plasmid-mediated but utilizes a chromosomal one-step alteration in the DNA-dependent RNA polymerase. In subjects using rifaximin no relevant resistance has been observed. Further, mutant resistant bacteria showed reduced viability and there is no systemic cross resistance for rifampin.

Since rifaximin is practically insoluble in water and is non absorbed (<0.4%) after oral administration, it can be used to treat localized diseases of the gastrointestinal tract. Rifaximin products specific for enteric pathogens of the gastro-intestinal tract are presently commercially marketed under various trade names —NORMIX® available from Alfa Wassermann S.p.A., Bologna, Italy; XIFAXAN® available from Salix Pharmaceutical, Raleigh, N.C.; REDACTIV® available from GlaxoSmithKline and FLONORM® from Schering-Plough. Since the solubility of rifaximin in water is approximately 1 μgmL³ the drug is virtually undissolved when traveling through the GI tract. The relative insolubility of rifaximin is thought to influence bacterial susceptibility and subsequent eradication due to the invasive nature of some enteric pathogens (e.g. Salmonella and Campylobacter). The relative insolubility of rifaximin also leads to its negligible systemic absorption. Rifaximin has been known to be effective for treating infections that are localized to the gut and is not known to be suitable for treating systemic infections caused by invasive organisms.

Rifaximin has been marketed in Italy since 1985 under the trademark NORMIX® for treating acute and chronic intestinal infections from gram-positive and gram-negative bacteria and as adjuvant in the therapy of the hyperammonoaemia. At present NORMIX® is marketed in the shape of pharmaceutical compositions, orally administrable, made by tablets or by granulates containing suitable pharmaceutically acceptable excipients together with rifaximin, but also other pharmaceutical forms orally administrable like capsules, sugar coated tablets and syrups can be used.

Xifaxan® is marketed in the United States and Canada and includes rifaximin as the active ingredient. The formulation is used in the treatment of travelers' diarrhea caused by the noninvasive strains of Escherichia coli. Xifaxan® is a non absorbable antiobiotic for gastrointestinal infections. Dr. Herbert DuPont, director of the Center for Infectious Diseases at the University of Texas, School of Public Health developed the drug for treatment of travelers' diarrhea. DuPont said “the drug is unique in that it remains in the gastrointestinal tract, compared with powerful antibiotics like Cipro that disperse throughout the body. This means the drug is less likely to breed resistant bacteria.” He said the antibiotic proved 85% effective in protecting US students who participated in a two-week study trip to Mexico, versus just 49% who didn't become sick on non-medicinal placebos. The drug has been found to have no significant side effects.

Products similar to NORMIX® and Xifaxan® are marketed in Mexico under the tradenames REDACTIV® and FLONORM®.

Other uses of rifaximin are disclosed in the following patents:

U.S. Pat. No. 5,886,002 to Ferrieri et al. describes use of rifaximin compositions in the treatment of diarrhea from cryptosporidiosis.

U.S. Pat. No. 5,352,679 to Ferrieri et al. describes use of rifaximin (INN) in formulations for treatment of gastric dyspepsia caused by Helicobacter pylori bacteria.

U.S. Pat. Nos. 5,314,904 and 6,140,355 both to Egidio et al. disclose compositions containing rifaximin for treatment of vaginal infections.

Known therapeutic uses of rifaximin, administered in a tablet form, include Clostridum difficile-associated diarrhea, Crohn's disease, Diverticular disease, Hepatic encephalopathy, Helicobacter pylon eradication, infectious diarrhea, irritable bowel syndrome, pouchitis, prophylaxis for GI surgery, small bowel overgrowth, traveler's diarrhea and ulcerative colitis. These therapies are directed to pediatric, adult and elderly subjects.

At present rifaximin has been studied and marketed only for the treatment of certain bacterial infections located in the gastrointestinal and reproductive tracts. However the prevention and treatment of upper respiratory illnesses such as pneumonia, bronchitis and tuberculosis has never been investigated.

Pneumonia is a lung infection that can be caused by different types of micro-organisms including bacteria, viruses, fungi, and parasites. It is a serious upper respiratory infection that is one of the leading causes of death in both the elderly, immuno-compromised and young. Death from the flu is usually from ensuing bacterial pneumonia and not from the flu virus. Use of current antibiotics to prevent ensuing pneumonia is complicated due to systemic effects, resistance, colonization with more virulent strains of bacteria and lack of efficacy.

It would be advantageous to have an antibiotic that was not systemically absorbed and could act locally to treat and prevent infection in the lungs and bronchial airways. This antibiotic could be administered to high risk patients to protect against pneumonia, bronchitis, tuberculosis and other upper respiratory infections to assist in saving many lives and reducing the morbidity of such illnesses.

Antibiotics, such as rifaximin, that are non-absorbed by the body, have not been used to treat or prevent an upper respiratory illness such as pneumonia, bronchitis or tuberculosis. The present invention provides advantage in doing so such that there are no significant systemic side effects. The invention preparations which contain rifaximin directly target the cause of the infection without causing systemic harm to the person.

In addition, rifaximin is an antibiotic with a broad spectrum of in vitro bactericidal activity, and as resistance is not mediated through plasmids, it is not transferable to other bacteria. If resistance did develop, bacteria would be substantially less able to become pathogenic as they could not produce the RNA dependent proteins as effectively.

Accordingly the present invention is directed to use of rifaximin in preparations to prevent and treat upper respiratory illnesses, such as pneumonia, bronchitis and tuberculosis. The present invention provides a method for delivering a non-systemic antibiotic, rifaximin, in an airborne form which is inhaled by a subject. Preferably, rifaximin is nebulized or prepared in a powder form which is inhaled by the intended subject. The rifaximin can also be used in conjunction with a broncodilator.

It is also contemplated by the invention, in patients that have already contracted an upper respiratory infection, the airborne rifaximin treatment could be combined and act synergistically with systemic antibiotics to treat the disease.

It is a general object of the invention to prevent and treat upper respiratory illnesses, such as pneumonia, bronchitis and tuberculosis, by treatment with airborne rifaximin formulations.

A further specific object of the invention is to prevent and treat pneumonia, bronchitis and tuberculosis by providing a nebulized form of rifaximin.

Another general object of the invention is to prevent and treat pneumonia, bronchitis and tuberculosis by further using the rifaximin formulations in conjunction with a broncodilator.

Another specific object of the invention is to treat and prevent pneumonia, bronchitis and tuberculosis by combining the airborne rifaximin treatment with a systemic antibiotic. The systemic antibiotic can be delivered in any manner such as orally, intravenously or nebulized.

### SUMMARY OF THE INVENTION

The present invention provides a method of preventing and treating upper respiratory illnesses which consists of airborne administration to a subject in need of such treatment a composition containing a therapeutically effective amount of a non-systemic antibiotic, preferably rifaximin.

The method of the invention treats and prevents upper respiratory illnesses that are caused by bacteria, protozoa, mycobacterium, RNA dependent viruses, reverse transcriptase dependent viruses and any other infections etiology that utilizes RNA.

Electrolytic reduction of rifaximin produces a slightly different structure referred to as rifaximin OR (open ring). As used in the specification herein, when the term rifaximin is used it is intended to also include the rifaximin OR structure unless otherwise stated. It is believed that the two molecules are similar in structure but have different chemical properties in solution.

In general, compositions containing a therapeutically effective amount of rifaximin are administered via airborne transmission to be inhaled by a subject in need of such treatment. The composition is preferably a pharmaceutical composition and contains a therapeutically effective amount of rifaximin which preferably delivers a dosage to achieve a concentration of up to 10,000 or more μg/ml per application. It is believed that dosages in the concentration range between 1-1000 μg/ml per application would also be effective. The duration of treatments with the invention formulations can be from one to three times per day to once a month depending on the individual and the desired outcome.

Rifaximin as a powder or solid granular form can be delivered to the subject in this form or can be is incorporated into a liquid preparation. Since the rifaximin essentially is non-reactive, it can be incorporated into aqueous or non-aqueous formulations without losing its efficacy. The composition can also be a dispersable or disintegrating tablet which maybe placed in solution for time released embodiments.

In all embodiments of the invention the compositions are made to be airborne so that they can be inhaled by the subject to prevent infection in the lungs and bronchial airways.

The compositions can be nebulized with or without a broncodilator. They can also be provided as an aerosol spray, mist, inhaled, dissolved or powder forms.

In addition, systemic antibiotics delivered orally, intravenously or inhaled, may be included in treatment depending on the condition and degree of illness be treated.

Rifaximin is preferably used, although other non-systemic antibiotics can be used and are within the scope of the invention.

Although this disclosure is directed to the preferred use of rifaximin, it is also within the scope of the invention that any non-systemic antibiotic can be included in the compositions and are included herein.

Other objects, features and advantages of the present invention will be apparent when the detailed description of the preferred embodiments of the invention are considered with reference to the drawings, which should be construed in an illustrative and not limiting sense as follows:

### BRIEF DESCRIPTION OF THE DRAWINGS

FIG. 1 is the chemical structure of rifaximin.

### DETAILED DESCRIPTION OF THE INVENTION

In general pneumonia is defined as an acute infection of lung parenchyma including alveolar spaces and interstitial tissue; involvement may be confined to an entire lobe (lobar pneumonia), a segment of a lobe (segmental or lobular pneumonia), alveoli contiguous to bronchi (bronchopneumonia) or interstitial tissue (interstitial pneumonia). These distinctions are generally based on x-ray observations. The etiology and the epidemiology are discussed—such as the types of bacteria responsible for pneumonia, predisposing factors and statistics involved with pneumonia

There are various types of pneumonia such as Pneumococcal Pneumonia, Staphylococcal Pneumonia, Streptococcal Pneumonia, pneumonia caused by Klebsiella Pneumoniae and other Gram-Negative Bacilli, pneumonia caused by Hemophilus Influenzae, Pneumonia of Legionnaries' Disease, Mycoplasmal Pneumonia, and Chylamydial Pneumonia

Pneumonia occurs in patients all age groups, but young children and the elderly, as well as immunocompromised and immune deficient patients, are especially at risk. Casual therapy is with systemic antibiotics.

The present invention, in contrast to known treatments, provides a method of treatment and prevention of upper respiratory illnesses which consists of airborne administration to a subject in need of such treatment a composition containing a therapeutically effective amount of a non-systemic antibiotic. Rifaximin is a preferred antibiotic used in the invention, although other non-systemic antibiotics are incorporated within the scope of this disclosure.

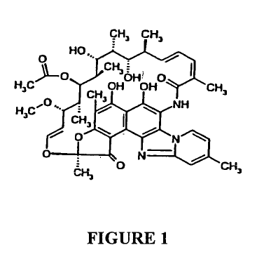

Rifaximin is a semi-synthetic, non-systemic antibiotic. The chemical name for rifaximin is (2S,16Z,18E,20S,21 S,22R,23R,24R,25S,26S,27S,28E)-5,6,21,23,25-pentahydroxy-27-methoxy-2,4,11,16,20,22,24,26-octamethyl-2,7-(epoxypentadeca-[1,11,13]trienimino)benzofuro[4,5-e]pyrido[1,2-α]-benzimidazole-1,15(2H)-dione,25-acetate. The empirical formula is C43H51N3O11 and its molecular weight is 785.9. The chemical structure is shown in FIG. 1.

The rifaximin preparations are effective against upper respiratory infections caused by bacteria such as pneumonia and bronchitis. In addition, they are effective against illnesses caused by mycobacterium such as tuberculosis. Tuberculosis is spread almost exclusively via the respiratory route. Exposure mandates treatment with long term toxic and in some cases life threatening drugs. The invention preparations are effective in treating people who have been exposed to tuberculosis (i.e. medical workers, family members) to receive airborne inhaled rifaximin as an adjunctive or alternative to systemic antibiotics.

They may also be effective against protozoa and RNA dependent viruses (i.e. HIV), reverse transcriptase dependent viruses and any other infections etiology that utilizes RNA. All the formulations are made to be airborne in a form which is inhaled by the subject to deliver the antibiotic to lung tissue.

Rifaximin is a semi-synthetic, non-systemic antibiotic. It acts by binding to the beta-subunit of bacterial DNA-dependent RNA polymerase thereby inhibiting RNA synthesis. It's lack of absorption reduces the chances of injuring the healthy bacteria that protect the body form infection (i.e. colliforms in the colon that protect from c-diff).

The invention formulations are formulated in either a nebulized preparation or a powder/disk form. In both instances the formulations are airborne and inhaled by the subject on a regular or intermittent basis. The formulations are directed to protect high risk individuals from pneumonia, bronchitis or tuberculosis as well as to treat patients with active pulmonary or bronchial infections. They may also be used in conjunction with other oral or intravenous antibiotics.

The airborne preparations of rifaximin can also be combined with any bronchiodilator to effectively deliver the antibiotic to the bronchioles and lung tissue.

The invention formulations may also be provided in an aerosol form.

Aerosol, nebulized or any airborne preparation of a non-absorbed antibiotic that can be inhaled would have the benefit of acting locally to prevent and help treat pneumonia, bronchitis or tuberculosis without having the complications of systemic resistance and side effects. The antibiotic would stay locally concentrated in the area that it is applied to and would not dilute into the systemic system of the individual. It would also be less likely to allow resistant strains to colonize in the individual subject.

By combining the invention preparations with systemic antibiotics the morbidity and mortality of upper respiratory infections could be substantially reduced by providing both local concentration of a synergistic antibiotic with a systemic antibiotic.

By combining the invention preparations with a bronchiodilator the antibiotic is better delivered to the bronchial airway and lung tissue.

As contemplated by the invention, the rifaximin can be delivered to the patient in various airborne forms. In general, in the compositions of the invention provide airborne delivery of a therapeutically effective amount of rifaximin to deliver a dosage to achieve a concentration of up to 10,000 or more μg/ml per application. It is believed that dosages in the concentration range between 1-1000 μg/ml per application would also be effective. The rifaximin preparations work on the surfaces to which they are exposed with essentially little to no absorption into the tissue itself. The duration of treatments with the invention formulations can be at daily, weekly or monthly intervals depending on the individual and the desired outcome.

These forms are now more particularly described.

In a first embodiment, pharmaceutical compositions, administrable by airborne delivery to the subject for inhalation, are made by providing a powder or granulates of a non-systemic antibiotic, preferably rifaximin, in a liquid preparation. Since the rifaximin is non-reactive, it can be incorporated into any liquid formulation without losing its efficacy. The liquid preparation can be placed in a nebulizer with or without a bronchiodialtor which is then inhaled by the subject.

In an alternate embodiment the preparation is made from a dispersable or disintegrating disc or powder which contains the non-systemic antibiotic. This solution is made airborne and is inhaled by the subject to distribute the antibiotic to the lungs and bronchial tissue.

The invention preparations can be further combined with a systemic antibiotic to help the subject overcome the upper respiratory infection. The systemic antibiotic can be delivered in any manner including airborne, oral or parientially.

The rifaximin preparations are used either on a continuous (i.e. daily, every other day, weekly, etc.) or intermittent basis to patients who have or are at risk for upper respiratory infections such as pneumonia, bronchitis and tuberculosis.

It is also contemplated within the invention, that the delivery methods and compositions according to the invention are useful in the prevention and treatment of upper respiratory infections due to exposure to spore forming bacteria, i.e. anthrax. In such treatments, airborne delivery of a therapeutically effective amount of rifaximin is provided to deliver a dosage to achieve a concentration greater than 10,000 or more μg/ml per application, preferably, concentrations greater than 20,000 to 30,000 μg/ml per application.

The present invention will be illustrated in more detail by the following examples without limiting the scope of the invention in any way.

### EXAMPLE 1

A trial study determining optimal dose and airborne delivery methods according to the invention in the prevention and treatment of pneumonia.

### EXAMPLE 2

A trial study evaluating various systemic antibiotics coupled with the invention airborne rifaximin preparations, nebulized or aerosol, to determine preferred combinations for treatment of upper respiratory illnesses. The systemic antibiotics are administered orally, inhaled and parientially.

### EXAMPLE 3

A trial study evaluating various types of bronchiodilators, to determine preferred combinations with rifaximin preparations in the treatment of upper respiratory illnesses.

### EXAMPLE 4

A trial study evaluating various doses and delivery methods according to the invention in the prevention and treatment of upper respiratory infections due to exposure to spore forming bacteria, i.e. anthrax.

The invention now being fully described, it will be apparent to one of ordinary skill in the art that many changes and modifications can be made thereto without departing from the spirit or scope of the invention as set forth herein.

What is claimed is:

1. A method of treatment and prevention of upper respiratory illnesses which consists of airborne administration to a subject in need of such treatment a composition containing a therapeutically effective amount of a non-systemic antibiotic.

2. A method according to claim 1 wherein the upper respiratory illnesses are caused by bacteria.

3. A method according to claim 1 wherein the upper respiratory illnesses are caused by protozoa, mycobacterium, RNA dependent viruses, reverse transcriptase dependent viruses and any other infections etiology that utilizes RNA.

4. A method according to claim 1, wherein said non-systemic antibiotic is rifaximin.

5. A method according to claim 1, wherein said composition is a pharmaceutical composition.

6. A method according to claim 1 wherein said therapeutically effective amount of said non-systemic antibiotic delivers a dosage to achieve a concentration of up to 10,000 or more μg/ml per application.

7. A method according to claim 1 wherein said therapeutically effective amount of said non-systemic antibiotic delivers a dosage to achieved a concentration between 1-1000 μg/ml per application.

8. A method according to claim 1 wherein said composition is inhaled by the subject to prevent infection in the lungs and bronchial airways.

9. A method according to claim 1 wherein said composition is a liquid preparation and is placed in a nebulizer and is inhaled by the subject.

10. A method according to claim 1 wherein said composition is a soluble or disintegrating tablet and is placed in a nebulizer and is inhaled by the subject.

11. A method according to claim 1 wherein said composition is placed in a bronchodilator.

12. A method according to claim 1 wherein said composition further contains a systemic antibiotic.

13. A method according to claim 1 wherein the upper respiratory illnesses are caused by spore forming bacteria and said therapeutically effective amount of said non-systemic antibiotic delivers a dosage to achieve a concentration of greater than 10,000 or more μg/ml per application.

14. A method according to claim 1 wherein said composition is a powder and is inhaled by the subject.
